(19) United States
(12) Patent Application Publication
(10) Pub. No.: US 2025/0288557 A1
(43) Pub. Date: Sep. 18, 2025

(54) FORMULATION AND METHOD FOR TREATMENT OF INFECTIONS

(71) Applicant: Veterinary Pharmacy Corporation, Saint Peter, MN (US)

(72) Inventors: William Anthony Soderlund, JR., Saint Peter, MN (US); Patrick Leon Soderlund, Saint Peter, MN (US); Patrick John Smith, Saint Peter, MN (US); Alyssa Louise Real, Saint Peter, MN (US)

(21) Appl. No.: 19/223,590

(22) Filed: May 30, 2025

Related U.S. Application Data
(63) Continuation of application No. PCT/US2024/013325, filed on Jan. 29, 2024.
(60) Provisional application No. 63/385,881, filed on Dec. 2, 2022.

(51) Int. Cl. A61K 31/40; A61K 9/08; A61K 31/405; A61K 31/4164; A61K 31/65; A61P 33/02

(52) U.S. Cl. CPC A61K 31/40; A61K 9/08; A61K 31/405; A61K 31/4164; A61K 31/65; A61P 33/02

(57) ABSTRACT

Formulations and methods for treatment of coccidia infections in an animal. The method includes obtaining a formulation of Statin (e.g., Atorvastatin), Indomethacin, Doxycycline, Cimetidine, Lincomycin, Sulfamethoxazole, Trimethoprim, Diclazuril, Salinomycin, Budesonide, Amprolium, Azithromycin, and/or any combination thereof; and providing the formulation to an animal.

### RELATED APPLICATIONS

This disclosure claims priority to and is a continuation application of international patent application number PCT/US2024/013325, filed on Jan. 29, 2024, which U.S. Provisional Application No. 63/385,881 filed on Dec. 2, 2022, which are herein incorporated by reference in their entirety.

### FIELD

This disclosure is related to treatments and managements of infections in animals.

### BACKGROUND

Coccidiosis infections in swine can be caused by Isospora suis. Isospora suis can infect piglets that are between 5-21 days of age. The infections can produce severe enteritis and diarrhea. Effects of the disease include macroscopic lesions, which are inflammations of the small intestine; and microscopic lesions, which cause damage to the villi of intestinal epithelial cells. When the cells in the villi become incapable of absorbing fluids, diarrhea results. Historical treatments of coccidia infections required daily dosing of compounds for the treatment of symptoms and did not directly attack the coccidia itself. This cumbersome way of dosage administration leads to poor adoption rates and usage in the industry.

### SUMMARY

A combination formulation is proposed for the methods of treatment for infections, such as for example, coccidiosis infections caused by Isospora suis, but not necessarily only limited thereto.

In some aspects, the techniques described herein relate to a method for treatment of coccidia infections in an animal, including: obtaining a formulation, wherein the formulation includes Statin (such as, for example, Atorvastatin), NSAID (such as, for example, Indomethacin), Tetracycline analogue (such as, for example, Doxycycline), H2 Receptor Antagonist (such as, for example, Cimetidine), Lincosamide (such as, for example, Lincomycin), Sulfonamide (such as, for example, Sulfamethaoxole), Diaminopyrimidines (such as, for example Trimehtoprim), or any combination thereof; providing the formulation to an animal.

In some aspects, the techniques described herein relate to a method for treatment of coccidia infections in an animal, including: obtaining a formulation, wherein the formulation includes Statin (such as, for example, Atorvastatin), Indomethacin, Doxycycline, Cimetidine, Lincomycin, Sulfamethaoxole, Trimehtoprim, or any combination thereof; providing the formulation to an animal.

In some aspects, the techniques described herein relate to a method, wherein the formulation is a solution.

In some aspects, the techniques described herein relate to a method, wherein the solution is an oil-based solution.

In some aspects, the techniques described herein include Statin of from 0.0011 mg/ml to 21.33 mg/ml of the solution.

In some aspects, the techniques described herein include Atorvastatin of from 0.0011 mg/ml to 21.33 mg/ml of the solution.

In some aspects, the techniques described herein include Cimetidine of from 0.0012 mg/ml to 240 mg/ml of the solution.

In some aspects, the techniques described herein include Indomethacin of from 0.00243 mg/ml to 48.53 mg/ml of the solution.

In some aspects, the techniques described herein include Doxycycline of from 0.004 mg/ml to 80 mg/ml of the solution.

In some aspects, the techniques described herein include Lincomycin of from 0.0758 mg/ml to 306 mg/ml of the solution.

In some aspects, the techniques described herein include Sulfamethaoxole of from 0.333 mg/ml to 6660 mg/ml of the solution.

In some aspects, the techniques described herein include Trimehtoprim of from 0.067 mg/ml to 1340 mg/ml of the solution.

In some aspects, the techniques described herein include Sulfamethaoxole of from 0.333 mg/ml to 6660 mg/ml of the solution; and Trimehtoprim of from 0.067 mg/ml to 1340 mg/ml of the solution.

In some aspects, the techniques described herein relate to a formulation for treating coccidia infections in an animal, wherein the formulation includes Statin (such as, for example, Atorvastatin), NSAID (such as, for example, Indomethacin), Tetracycline analogue (such as, for example, Doxycycline), H2 Receptor Antagonist (such as, for example, Cimetidine), Lincosamide (such as, for example, Lincomycin), Sulfonamide (such as, for example, Sulfamethaoxole), Diaminopyrimidines (such as, for example Trimehtoprim), or any combination thereof.

In some aspects, the techniques described herein relate to a formulation for treating coccidia infections in an animal, wherein the formulation includes Statin, Indomethacin, Doxycycline, Cimetidine, Lincomycin, Sulfamethaoxole, Trimehtoprim, or any combination thereof.

In some aspects, the techniques described herein relate to a formulation for treating coccidia infections in an animal, wherein the formulation includes Atorvastatin, Indomethacin, Doxycycline, Cimetidine, Lincomycin, Sulfamethaoxole, Trimehtoprim, or any combination thereof.

In some aspects, the techniques described herein relate to a formulation, further including a solution.

In some aspects, the techniques described herein relate to a formulation, wherein the solution is an oil-based solution.

In some aspects, the techniques described herein relate to a formulation, wherein the Statin is from 0.0011 mg/ml to 21.33 mg/ml of the solution.

In some aspects, the techniques described herein relate to a formulation, wherein the Atorvastatin is from 0.0011 mg/ml to 21.33 mg/ml of the solution.

In some aspects, the techniques described herein relate to a formulation, wherein the Cimetidine is from 0.0012 mg/ml to 240 mg/ml of the solution.

In some aspects, the techniques described herein relate to a formulation, wherein the Indomethacin is from 0.00243 mg/ml to 48.53 mg/ml of the solution.

In some aspects, the techniques described herein relate to a formulation, wherein the Doxycycline is from 0.004 mg/ml to 80 mg/ml of the solution.

In some aspects, the techniques described herein relate to a formulation, wherein the Lincomycin is from 0.0758 mg/ml to 306 mg/ml of the solution.

In some aspects, the techniques described herein relate to a formulation, wherein the Sulfamethaoxole is from 0.333 mg/ml to 6660 mg/ml of the solution.

In some aspects, the techniques described herein relate to a formulation, wherein the Trimehtoprim is from 0.067 mg/ml to 1340 mg/ml of the solution.

In some aspects, the techniques described herein relate to a formulation, wherein the Sulfamethaoxole is from 0.333 mg/ml to 6660 mg/ml of the solution; and Trimehtoprim is from 0.067 mg/ml to 1340 mg/ml of the solution.

### DETAILED DESCRIPTION

Proposed treatment methods include treatments for infection(s) caused by, for example, Cryptosporidium parvum, Isopora suis, etc.

One or more of the following have been identified, and in some embodiments, various combinations of formulations have been identified as treatment methods for infection(s).
1. Statin (e.g., Atorvastatin)

Mechanism of action (MOA): Screening against Cryptosporidium parvum showed that IPP (Isopentenyl-5-pyrophosphate) is an essential metabolite in a parasite's life cycle. Blocking affects all down chain biosynthesis pathways. Cholesterol was also seen as a needed nutrient in the Cryptosporidium parvum life cycle.
II. NSAID (e.g., Indomethacin)

MOA: Shown to be active against Cryptosporidium parvum in HIV screening. Inhibits PGE2 synthesis which can increase IL-2 production. IL-2 can help stimulate and activate T cell host immune response system. PG levels are often associated with parasite induced immunosuppression. Promotion of local leukocyte migration into inflamed tissues.
III. Tetracycline analog (e.g., Doxycycline)

MOA: Tetracycline analog theorized to act directly on the Apicomplexa to damage DNA/RNA synthesis of the protozoa. Will also target opportunistic secondary infections that can weaken immune system. Additionally, Doxycycline can provide a secondary bacterial coverage as well to limit the impact of opportunistic infections.
IV. H2RA (e.g., Cimetidine)

MOA: Chemical Structure may lend itself to protozoal activity, synergism with increasing effect of other administered drugs, altered stomach pH to affect the breakdown of the oocyst in acidic environment. It is also a potent inhibitor of metabolism of the above drugs which was theorized to synergistically increase therapeutic effect.
V. Lincosamide antibiotic (e.g., Lincomycin)

MOA: Bacterial mechanism of action to reduce secondary infections in piglets experiencing severe diarrhea. The antibacterial action of lincomycin is the ability of the drug to bind to the 50s ribosomal unit and inhibit protein synthesis. Lincosamides like Clindamycin and Lincomycin are also inferred to reduce shedding of protozoal species.
VI. Sulfamethoxazole and Trimethoprim (Sulfa/TMP, Sulfa antibiotic)

MOA: Sulfamethoxazole, a sulfonamide, induces its therapeutic effects by interfering with the de novo (that is, from within the cell) synthesis of folate inside microbial organisms such as protozoa, fungi and bacteria. It does this by competing with p-aminobenzoic acid (PABA) in the biosynthesis of dihydrofolate. Trimethoprim serves as a competitive inhibitor of dihydrofolate reductase (DHFR), hence inhibiting the de novo synthesis of tetrahydrofolate, the biologically active form of folate.
VII. Diclazuril (Nucleoside analog)

MOA: Although the mode of action of diclazuril is not precisely known, its effect on the asexual or sexual stages of coccidies blocks the excretion of oocysts inducing an interruption of the life cycle of the parasites.
VIII. Toltatruzil (triazinetrione derivative)

MOA: Induces changes in the fine structure of coccidian development stages that are mainly due to a swelling of the endoplasmic reticulum and of the Golgi apparatus and to abnormalities in the peri-nuclear space, disturbances in nuclear division. Toltrazuril leads to a reduction of enzymes of the respiratory chain of the parasites.
IX. Ponazuril (triazine coccidiocidal drug that is related to toltrazuril)

MOA: Triazine antiprotozoals are considered to be active against the plastid body of the parasite. Plastids originated as a site for photosynthesis in plants. Although they have lost this function in apicomplexans, the plastid remains an essential organelle to those parasites that have them.
X. Salinomycin (lonophores)

MOA: Disturb normal transport of ions across the surface membranes of sporozoites and/or early merozoites
XI. Budesonide

MOA: Orally administered steroid that is not absorbed but rather acts locally on the intestinal tract. It is expected that it can help reduce inflammation caused by the parasite to the intestine (commonly used in Crohn's disease in adults and pediatrics) enhancing the activity of Indomethacin.
XII. Amprolium (Quartenerized derivative of pyrimidine with demonstrated activity against protozoal species including some strains of coccida)

MOA: It is a thiamine antagonist and due to its close structural similarity, it blocks the thiamine receptors. This blockage of receptors prevents coccidia from utilizing thiamine and as a result thiamine is unavailable to coccidian (Competitive inhibition of thiamine uptake). This vitamin (thiamine pyrophosphate) is a cofactor of several decarboxylase enzymes which play role in cofactor synthesis. At higher doses, thiamine deficiency can occur in host, but it can be prevented by addition of thiamine.
XIII. Azithromycin (Macrolide)

MOA: Inhibition of bacterial protein synthesis, inhibition of proinflammatory cytokine production, inhibition of neutrophil infestation, and macrophage polarization alteration, gives it the ability to act against a wide range of microorganisms. It is believed that it can be useful in treatment of infections that are parasitic or protozoal in nature.

According to some embodiments, a formulation including Statin (e.g., Atorvastatin), Indomethacin, Doxycycline, Cimetidine, Indomethacin, Doxycycline, Cimetidine, Lincomycin, Sulfamethoxazole and Trimethoprim, Diclazuril, Toltatruzil, Ponazuril, Salinomycin, Budesonide, Amprolium, Azithromycin, and/or any combinations thereof, is provided and/or used to treat coccidia infections.

The any one or any combination(s) of the four drugs provide varying mechanisms of action and have longer half-lives. The drugs can interact with each other to increase duration in the bodies system to allow for a reduction of dosing requirements (e.g., 1-2-time dosing).

### Example Drug Formulation

a. To administer this drug in a uniform form that is easily deliverable, the following formulation was chosen. This is an oil-based delivery method (e.g., using medium chain triglyceride (MCt) oil, coconut oil, and/or corn oil). The formulation has been tested using dosage guns and examine for caking and found to be able to be easily resuspended after prolonged settling.
b. Active Ingredients to make 1000 dose batch

| | | | |
|---|---|---|---|
| i. | Atorvastatin Calcium | 16 | g |
| ii. | Cimetidine | 18 | g |
| iii. | Indomethacin | 3.64 | g |
| iv. | Doxycycline Hyclate | 6 | g |
| v. | Colloidal Silicon Dioxide | 10 | g |
| vi. | Simethicone Defoamer | 1.3 | ml |
| vii. | Cherry Flavor | 1.47 | g |

| | | |
|---|---|---|
| viii. | Oil | Q.S. to 1000 ml |

Safety Study: Studies for safety were conducted. The trials assessed the active ingredients individually and according to various combinations.

Summary: No signs of toxicity or adverse events were found. Data indicate that the formulations and methods are effective at treatment of and preventing coccidia in the pigs with no indication of side effects. There were no health concerns post-administration of the active ingredients.

Example Safety Study: The following provides the procedure(s) and data collected from the exemplary safety study.

Six different sets of pigs were tested with six different formulations:
1. Negative control (no active ingredients/i.e., no drugs)
2. Statin or Atorvastatin (1.6 mg/dose)
3. Indomethacin (3.64 mg/dose)
4. Doxycycline (6 mg/dose)
5. Cimetidine (18 mg/dose)
6. Positive control (compounded formulation of Atorvastatin, Indomethacin, Indomethacin, Doxycycline, and Cimetidine)

Methods: A sow farm negative for Porcine Reproductive and Respiratory Syndrome virus (PRRSv) and Mycoplasma hyopneumoniae and experiencing little to no piglet enteric health challenges was enrolled for the study. Piglets (12-14 days of age) orally received one of the six formulations identified above. When receiving a formulation, 1 mL of assigned treatment was obtained and provided to the Piglets.

Data collection: Clinical observations suggestive of product(s) side-effects (i.e., vomiting, muscle spasms, lameness, ataxia) were individually recorded per pig prior to do (day 0) and d1 (i.e., 24 hours or day 1) post-administration. Moreover, clinical signs suggestive of poor palatability (e.g., pharyngeal (gag) reflex, expectoration, etc.) were recorded post-administration of products. At d0 and d1, pen-level fecal consistency was studied. At d1, piglet mortality was recorded.

Results: Twelve litters were enrolled with a total of 153 pigs, averaging 12.7 pigs per litter. Average dam parity was 3.1. Table 1 depicts farrowing characteristics, clinical observations, average fecal scores, and mortality per treatment. At d 0 and d 1, clinical signs suggestive of product(s) side-effects were not observed, regardless of treatment. Moreover, signs suggestive of poor palatability were not observed post-product administration. Prior to administration (d 0), normal fecal consistency scores (score 0) were observed, except for an Atorvastatin-treated litter that had a fecal score of 1. At d 1, differences in fecal consistency scores were not observed and all litters had normal fecal composition. Additionally, 6.7% mortality ( 1/25) was observed for litters that received the compounded formulation (positive control), in which the cause of death was deemed as a ‘laid on’ and not the result of the administered product.

TABLE 1

| | Negative | | | | | Positive |
|---|---|---|---|---|---|---|
| | control | Atorvastatin | Indomethacin | Doxycycline | Cimetidine | control |
| Farrowing | | | | | | |
| characteristics | | | | | | |
| Parity (avg) | 3.0 | 5.0 | 3.0 | 2.0 | 2.5 | 3.5 |
| # pigs | 26 | 25 | 26 | 25 | 26 | 25 |
| enrolled | | | | | | |
| Pigs/litter | 13.0 | 12.5 | 13.0 | 12.5 | 13.0 | 12.5 |
| (avg) | | | | | | |
| Clinical | | | | | | |
| observations | | | | | | |
| (%) | | | | | | |
| d 0 | 0.0 | 0.0 | 0.0 | 0.0 | 0.0 | 0.0 |
| d 1 | 0.0 | 0.0 | 0.0 | 0.0 | 0.0 | 0.0 |
| Fecal scores | | | | | | |
| d 0 | 0.0 | 0.5 | 0.0 | 0.0 | 0.0 | 0.0 |
| d 1 | 0.0 | 0.0 | 0.0 | 0.0 | 0.0 | 0.0 |
| Palatability | | | | | | |
| (%) | | | | | | |
| d 0 | 0.0 | 0.0 | 0.0 | 0.0 | 0.0 | 0.0 |
| Mortality (%) | | | | | | |
| d 0 | 0.0 | 0.0 | 0.0 | 0.0 | 0.0 | 0.0 |
| d 1 | 0.0 | 0.0 | 0.0 | 0.0 | 0.0 | 6.7 |

Clinical observations: Percentage of pigs expressing clinical signs suggestive of product(s) side-effects (i.e., vomiting, muscle spasms, lameness, ataxia).

Fecal scores: Average fecal score (pen-level). Fecal scours 0, 1, 2, and 3 corresponded to normal, pasty, semi-liquid, and liquid feces, respectively, with scores of ≥2 corresponding to diarrhea.

Palatability: Percentage of pigs expressing clinical signs suggestive of poor palatability (e.g., pharyngeal (gag) reflex, expectoration).

Mortality: %=(number of dead pigs/number of pigs enrolled)×100.

Conclusions: Clinical signs suggestive of product side-effects and mortality attributed to the antiparasitic producers were not observed, resulting in no health concerns post-administration and suggesting product(s) safety in piglets.

Additional tests were performed on pigs, wherein about 750,000 doses of formulations were administered. Some results indicated about 12-22% reduction in Scours (Diarrhea). In an exemplary study, the result was 42% reduction in Scours. In an exemplary study, the result was 45.4% reduction in Scours. In another exemplary study, the result was 16% reduction in Scours. According to some embodiments, the reduction in Scours has a range of 12%-45.4% reduction. According to some embodiments, the reduction in Scours has a range of 16%-45.4% reduction. According to some embodiments, the reduction in Scours has a range of 22%-45.4% reduction. According to some embodiments, the reduction in Scours has a range of 42%-45.4% reduction. According to some embodiments, the reduction in Scours has a range of 12%-16% reduction. According to some embodiments, the reduction in Scours has a range of 12%-22% reduction. According to some embodiments, the reduction in Scours has a range of 12%-42% reduction. According to some embodiments, the reduction in Scours has a range of 16%-42% reduction. According to some embodiments, the reduction in Scours has a range of 16%-22% reduction. According to some embodiments, the reduction in Scours has a range of 22%-42% reduction.

According to some embodiments, the formulation includes Atorvastatin, at for example 1.6 mg/1.5 ml.

According to some embodiments, the formulation includes Cimetidine, at for example 18 mg/1.5 ml.

According to some embodiments, the formulation includes Indomethacin, for example 3.64 mg/1.5 ml.

According to some embodiments, the formulation includes Doxycycline, for example 6 mg/1.5 ml.

According to some embodiments, the formulation includes Lincomycin, for example 113.63 mg/1.5 ml.

According to some embodiments, the formulation includes colloidal silicone dioxide (at for example 0.026 g/1.5 ml), simethicone defoamer (at for example 0.019 g/1.5 ml), or both.

According to some embodiments, cherry flavor can also be added (at for example 0.002 g/1.5 ml).

According to some embodiments, BHT preservatives can also be added (at for example 0.0015 g/1.5 ml).

According to some embodiments, the formulation can include a brown colorant (at for example 0.0003 g/1.5 ml).

According to some embodiments, the formulation includes corn oil (Q.S. to final volume).

According to some embodiments, the formulation includes Statin, Cimetidine, Indomethacin, Doxycycline, Lincomycin, Sulfamethaoxole, Trimehtoprim, and/or any combination thereof.

According to some embodiments, the formulation includes Atorvastatin, Cimetidine, Indomethacin, Doxycycline, and Lincomycin.

According to some embodiments, the formulation includes Atorvastatin, at for example 1.6 mg/1.5 ml; Cimetidine, at for example 18 mg/1.5 ml; Indomethacin, for example 3.64 mg/1.5 ml; Doxycycline, for example 6 mg/1.5 ml; and Lincomycin, for example 113.63 mg/1.5 ml.

According to some embodiments, the formulation includes Atorvastatin, at for example 1.6 mg/1.5 ml; Cimetidine, at for example 18 mg/1.5 ml; Indomethacin, for example 3.64 mg/1.5 ml; and Doxycycline, for example 6 mg/1.5 ml.

According to some embodiments, the formulation includes Atorvastatin, at for example 1.6 mg/1.5 ml; Cimetidine, at for example 18 mg/1.5 ml; and Indomethacin, for example 3.64 mg/1.5 ml.

According to some embodiments, the formulation includes Atorvastatin, at for example 1.6 mg/1.5 ml; and Cimetidine, at for example 18 mg/1.5 ml.

According to some embodiments, the formulation includes:
1.) Statin or Atorvastatin 1.6 mg/1.5 ml
2.) Cimetidine 18 mg/1.5 ml
3.) Indomethacin 3.64 mg/1.5 ml
4.) Doxycycline 6 mg/1.5 ml
5.) Lincomycin 113.63 mg/1.5 ml
6.) Colloidal Silicone Dioxide 0.026 g/1.5 ml
7.) Simethicone Defoamer 0.019 g/1.5 ml
8.) Cherry Flavor 0.002 g/1.5 ml
9.) BHT Preservative 0.0015 g/1.5 ml
10.) Brown Colorant 0.0003 g/1.5 ml
11.) Corn Oil Q.S. to final volume

In some embodiments, the active ingredients in the formulation or combinations of formulations can be substituted by similar compositions having the same or similar MOA.

### Aspects

Aspect 1. A method for treatment of coccidia infections in an animal, comprising: obtaining a formulation, wherein the formulation includes Statin (e.g., Atorvastatin), Indomethacin, Doxycycline, Cimetidine, Lincomycin, Sulfamethoxazole, Trimethoprim, Diclazuril, Toltatruzil, Ponazuril, Salinomycin, Budesonide, Amprolium, Azithromycin, or any combination thereof; and providing the formulation to an animal.
Aspect 2. The method of Aspect 1, wherein the formulation is a solution.
Aspect 3. The method of Aspect 2, wherein the solution is an oil-based solution.
Aspect 4. The method according to any of Aspect 1 to 3, wherein the Statin is from 0.0011 mg/ml to 21.33 mg/ml of the solution.
Aspect 5. The method according to any of Aspect 1 to 4, wherein the Cimetidine is from 0.0012 mg/ml to 240 mg/ml of the solution.
Aspect 6. The method according to any of Aspect 1 to 5, wherein the Indomethacin is from 0.00243 mg/ml to 48.53 mg/ml of the solution.
Aspect 7. The method according to any of Aspect 1 to 6, wherein the Doxycycline is from 0.004 mg/ml to 80 mg/ml of the solution.
Aspect 8. The method according to any of Aspect 1 to 7, wherein the Lincomycin is from 0.0758 mg/ml to 306 mg/ml of the solution.
Aspect 9. The method according to any of Aspect 1 to 8, wherein the Sulfamethaoxole is from 0.0758 mg/ml to 306 mg/ml of the solution.
Aspect 10. The method according to any of Aspect 1 to 9, wherein the Trimehtoprim is from 0.067 mg/ml to 1340 mg/ml of the solution.
Aspect 11. A formulation for treating coccidia infections in an animal, comprising: Statin (e.g., Atorvastatin), Indomethacin, Doxycycline, Cimetidine, Indomethacin, Doxycycline, Cimetidine, Lincomycin, Sulfamethoxazole and Trimethoprim, Diclazuril, Toltatruzil, Ponazuril, Salinomycin, Budesonide, Amprolium, Azithromycin, and/or any combination thereof.
Aspect 12. The formulation of Aspect 11, further comprising a solution.
Aspect 13. The formulation of Aspect 12, wherein the solution is an oil-based solution.
Aspect 14. The formulation according to any of Aspect 11 to 13, wherein the Statin is from 0.0011 mg/ml to 21.33 mg/ml of the solution.
Aspect 15. The formulation according to any of Aspect 11 to 14, wherein the Cimetidine is from 0.0012 mg/ml to 240 mg/ml of the solution.
Aspect 16. The formulation according to any of Aspect 11 to 15, wherein the Indomethacin is from 0.00243 mg/ml to 48.53 mg/ml of the solution.
Aspect 17. The formulation according to any of Aspect 11 to 16, wherein the Doxycycline is from 0.004 mg/ml to 80 mg/ml of the solution.
Aspect 18. The formulation according to any of Aspect 11 to 17, wherein the Lincomycin is from 0.0758 mg/ml to 306 mg/ml of the solution.
Aspect 19. The formulation according to any of Aspect 11 to 18, wherein the Sulfamethaoxole is from 0.0758 mg/ml to 306 mg/ml of the solution.
Aspect 20. The formulation according to any of Aspect 11 to 19, wherein the Trimehtoprim is from 0.067 mg/ml to 1340 mg/ml of the solution.

What is claimed is:

1. A method for treatment of coccidia infections in an animal, comprising:
obtaining a formulation,
wherein the formulation includes Statin, Atorvastatin, Indomethacin, Doxycycline, Cimetidine, Lincomycin, Sulfamethoxazole, Trimethoprim, Diclazuril, Salinomycin, Budesonide, Amprolium, Azithromycin, or any combination thereof; and
providing the formulation to an animal.

2. The method of claim 1, wherein the formulation is a solution.

3. The method of claim 2, wherein the solution is an oil-based solution.

4. The method according to claim 1, wherein the Statin is from 0.0011 mg/ml to 21.33 mg/ml of the solution.

5. The method according to claim 1, wherein the Cimetidine is from 0.0012 mg/ml to 240 mg/ml of the solution.

6. The method according to claim 1, wherein the Indomethacin is from 0.00243 mg/ml to 48.53 mg/ml of the solution.

7. The method according to claim 1, wherein the Doxycycline is from 0.004 mg/ml to 80 mg/ml of the solution.

8. The method according to claim 1, wherein the Lincomycin is from 0.0758 mg/ml to 306 mg/ml of the solution.

9. The method according to claim 1, wherein the Sulfamethaoxole is from 0.0758 mg/ml to 306 mg/ml of the solution.

10. The method according to claim 1, wherein the Trimehtoprim is from 0.067 mg/ml to 1340 mg/ml of the solution.

11. A formulation for treating coccidia infections in an animal, comprising:
Statin, Atorvastatin, Indomethacin, Doxycycline, Cimetidine, Lincomycin, Sulfamethoxazole, Trimethoprim, Diclazuril, Salinomycin, Budesonide, Amprolium, Azithromycin, and/or any combination thereof.

12. The formulation of claim 11, further comprising a solution.

13. The formulation of claim 12, wherein the solution is an oil-based solution.

14. The formulation according to claim 11, wherein the Statin is from 0.0011 mg/ml to 21.33 mg/ml of the solution.

15. The formulation according to claim 11, wherein the Cimetidine is from 0.0012 mg/ml to 240 mg/ml of the solution.

16. The formulation according to claim 11, wherein the Indomethacin is from 0.00243 mg/ml to 48.53 mg/ml of the solution.

17. The formulation according to claim 11, wherein the Doxycycline is from 0.004 mg/ml to 80 mg/ml of the solution.

18. The formulation according to claim 11, wherein the Lincomycin is from 0.0758 mg/ml to 306 mg/ml of the solution.

19. The formulation according to claim 11, wherein the Sulfamethaoxole is from 0.0758 mg/ml to 306 mg/ml of the solution.

20. The formulation according to claim 11, wherein the Trimehtoprim is from 0.067 mg/ml to 1340 mg/ml of the solution.
